(19) United States
(12) Patent Application Publication
(10) Pub. No.: US 2025/0229069 A1
(43) Pub. Date: Jul. 17, 2025

(54) Support for Microneedle Array

(71) Applicant: LTS Lohmann Therapie-Systeme AG, Andernach (DE)

(72) Inventors: Stefan Erlhofer, Ochtendung (DE); Sebastian Scherr, Neuhäusel (DE); Michael Kulik, Urmitz (DE)

(21) Appl. No.: 19/095,589

(22) Filed: Mar. 31, 2025

Related U.S. Application Data
(62) Division of application No. 17/423,653, filed on Jul. 16, 2021.

(30) Foreign Application Priority Data
Jan. 17, 2019 (DE) 102019200561.6

(51) Int. Cl. A61M 37/00

(52) U.S. Cl. CPC A61M 37/0015; A61M 2037/0023; A61M 2037/0046; A61M 2037/0053

(57) ABSTRACT

A support for at least one microarray, with a housing and a punch arranged in a movable manner within the housing. At least one microarray is connected to the punch. The housing includes an application opening for applying the at least one microarray. Further, a method for producing a support for at least one microarray. The method includes the steps of manufacturing a housing with an application opening, manufacturing a punch, connecting a microarray to the punch, and arranging the punch in a movable manner within the housing.

### CROSS-REFERENCE TO RELATED APPLICATIONS

This application is a divisional of U.S. patent application Ser. No. 17/423,653 filed Dec. 19, 2019, which is the United States national phase of International Application No. PCT/EP2019/086372 filed Dec. 19, 2019, and claims priority to German Patent Application No. 102019200561.6 filed Jan. 17, 2019, the disclosures of which are hereby incorporated by reference in their entirety.

### BACKGROUND OF THE DISCLOSURE

### Field of the Disclosure

The disclosure relates to a support for at least one microarray and to a method for producing a support for at least one microarray.

### Description of Related Art

Microarrays have a multiplicity of microneedles which usually are arranged on a support element, such as a patch, a plaster or the like, or are connected to a support element. Such microarrays have a high number of microneedles, of for example 500-600 needles per cm². The needles are of short length, so that when the microneedles are pressed into a patient's skin the needles penetrate only so far into the skin that nerves and blood vessels as far as possible are not contacted by needle tips. The microneedles contain an active ingredient or a medicament. The corresponding active ingredient may be applied to a surface of the needle or be arranged in the needles. It is preferred for the needles to be produced from a material which dissolves in the patient's skin.

Numerous problems arise in the handling of microarrays.

For example, sterile transport is required, as is sterile storage of the microarrays. This is necessary in order on one hand to protect the active ingredients to be administered with the microarrays from environmental influences. On the other hand, it is necessary to prevent contamination of the microarrays, so that no contamination of the application points occurs when applying the microarrays.

Furthermore, problems arise with regard to the microneedles of the microarrays. These are susceptible to physical action, as a result of which damage and ultimately failure of the microneedles may occur. Since microneedles are frequently made soluble, it happens that the needles are damaged in contact with liquids or vapor as well. Further still, with exposed microneedles unintentional injection of people, for example medical personnel, may occur.

The above-mentioned problems also occur with conventionally-packaged microarrays in the time between unpacking and application.

More extensive problems arise due to the necessity of applying microarrays over a longer timeframe. For example, at present there are none, or merely inadequate, designs for lasting application of microarrays. Furthermore, in the case of lasting application too problems again arise due to environmental influences, in particular with regard to the sterility of the application point.

### SUMMARY OF THE DISCLOSURE

The object of the disclosure is to provide a support for at least one microarray with which the handling of the microarray is improved. An independent further object of the disclosure is to provide a method for producing a support for at least one microarray wherein assembly is improved.

These objects are achieved according to the disclosure by a support having the features of claim 1 and a method for producing a support having the features of claim 14.

The support according to the disclosure for at least one microarray is in particular a packaging for at least one microarray. Alternatively or additionally, the support may also be a cartridge for at least one microarray. “Cartridge” in this case means a device which on one hand serves for enveloping, preferably packaging, at least one microarray and on the other hand during application is placed on the application point, in particular into the skin of a user, and accordingly preferably serves as a device for application. This device for application is embodied in particular in the manner of a patch of an insulin pump. The support according to the disclosure comprises a housing. The housing is in particular of cylindrical configuration. Preferably the cylinder shape has a circle or a rectangle, in particular a square, as its base. Thus the housing preferably has substantially the form of a regular cylinder or a cuboid, preferably a cube. Furthermore, the support comprises a punch arranged in a movable manner within the housing. The punch is in particular linearly, preferably axially, movable. It is particularly preferred for the punch to be distally movable. “Distally” designates the direction towards the application point, in particular perpendicularly towards the application point. “Proximally” accordingly designates the opposite direction. At least one microarray is connected to the punch. It is preferred for the at least one microarray to be connected to the punch at the distal end of the punch. The connection of the punch and the at least one microarray preferably takes place by means of an adhesive join, with in particular a patch of the microarray which bears the microstructures, preferably the microneedles, being adhesively connected to the punch. Alternatively, a one-piece, also referred to as integral, configuration composed of the punch and the at least one microarray is also possible. In the starting position of the punch, in which the punch is preferably in a proximal position, the at least one microarray is located preferably within the housing, with the at least one microarray in this case being stored in particular in a sterile manner. The housing comprises an application opening for applying the at least one microarray. Preferably the application opening is located at the distal end of the housing. The application opening preferably makes possible movement of the microarray, particularly preferably of the punch, at least partly out of the housing.

One advantage of the support is that it serves in particular for packaging, preferably dimensionally stable packaging, of at least one microarray, for example for transport and/or storage. On the other hand, it is in particular advantageously possible by means of the support to apply the at least one microarray directly with the support. It is particularly advantageous that by means of the support according to the disclosure a device is provided which makes possible simplified and preferably sterile handling during transport, storage and likewise during application.

In a preferred embodiment, the housing comprises a connection device for connecting to an applicator. The applicator to be connected is preferably an applicator for moving, in particular accelerating, the punch. To this end, it is preferred for the applicator to act on the punch by means of an applicator punch, such as for example a piston, or the like. The connection device can be preferably selectively coupled, so that on one hand a detachable connection is possible. On the other hand, it is preferably possible for it to be a non-detachable connection device, so that a permanent connection exists.

It is preferred for the connection device to have a groove and/or a bead and/or a thread and/or a plug-type connection and/or a form-fit connecting piece and/or an adhesive join and/or a magnetic connecting piece, in particular a magnet. In such a manner, an applicator having a corresponding counter-part can be connected to the housing of the support by way of the connecting device.

The housing preferably comprises an access opening for an applicator for moving the punch. This access opening is arranged in particular at the proximal end of the housing, particularly preferably opposite the application opening. In particular, it is possible to act on the punch by means of a piston or the like of the applicator by way of the access opening.

It is preferred for the housing and/or the punch to be at least substantially dimensionally stable. “Dimensionally stable” in this case means in particular a rigid and/or stiff configuration of the housing and/or punch. In addition to the substantially dimensionally stable configuration, it is possible in particular for the housing and/or punch to have flexible elements. The dimensionally stable configuration of the housing makes it possible to protect the microarray from in particular physical environmental influences. The dimensionally stable configuration of the punch makes possible optimum guidance and/or storage of the punch within the housing.

Preferably the support comprises a release device, in particular between the housing and punch. The release device is in particular embodied such that it fixes the punch in a starting position and releases it for movement upon activation of the release device. It is particularly preferred for the punch to be fixed in a proximal starting position. The activation of the release device preferably takes place by actuation by a user.

Preferably the release device comprises a desired-force trigger, the desired-force trigger releasing the punch for movement when at least one, in particular predetermined, desired force of the desired-force trigger is applied. The desired-force trigger comprises in particular a predetermined breaking point and/or a fit and/or a detachable form-fit connection, preferably between the housing and punch. The predetermined breaking point particularly preferably has a fragile supporting structure between the housing and punch. The supporting structure is in particular a supporting structure which has been produced by means of 3D printing. Preferably it is a supporting structure which has been created by joint production by 3D printing of the housing and the punch, with preferably the punch having been printed directly within the housing. Preferably the supporting structure has fragile connecting struts. The form-fit connection preferably has an in particular flexible groove and/or an in particular flexible bead. The form-fit connection is detachable, preferably by application of force. It is preferred for the punch to have a bead and the housing a groove, with the bead being arranged within the groove in the starting position and with this connection being overcome and the punch being movable upon application of at least the desired force to the punch. It is particularly preferred for the desired-force trigger to be triggered if at least the desired force is applied to the punch. It is preferred for the punch, after being released by the release device, no longer to be connected to the housing and/or to be loose from the housing, so that the housing and punch can be moved independently relative to each other. For example, the housing, after application of the microarray, which is connected to the punch, can be removed from the application point, while the microarray and/or punch remain(s) on the application point. It is preferred for the desired-force trigger to have a bistable spring element. With the bistable spring element it is preferred that, after at least the desired force is applied, it flips into a second bistable position due to stored energy and, in particular by a contacting action, moves the punch distally.

In a preferred embodiment, the support has a holding device for fixing the punch after the release of movement. The holding device has in particular an engagement device and/or a snap-in device and/or a fit and/or a bonded joint and/or a form-fit connection. The engagement device may preferably be an engagement hinge. The snap-in device is preferably a snap fastener. The holding device fixes the punch in particular in a distal position, the distal position being in particular the application position of the punch in which the at least one microarray is applied. The holding device makes possible in particular subsequent pressing and thus lasting application of the at least one microarray which is connected to the punch.

It is preferred for the support to have a fixing device for fixing the support to an application point. Preferably the fixing of the housing takes place at the distal end, in particular a distal base of the housing. If the fixing device has an adhesive surface, it is preferred for the distal face of the housing to have the adhesive. Alternatively or additionally, it is possible for the fixing device to have a loop and/or a tab, the loop and/or the tab being able to be tied for example around the arm or leg of a user and in such a manner fixing of the support to the application point taking place.

In a preferred embodiment, the support has a sterile barrier for sterile storage of the microarray. In particular, it is a sterile barrier for closing the application opening and/or the access opening for isolation of the interior of the housing from the surroundings. In such a manner, in particular the advantage is achieved that the microarray is stored in a sterile manner within the support.

The sterile barrier preferably comprises a film, wherein the film is arranged in particular at the access opening. It is preferred for the film to be connected to a proximal face of the housing. Preferably the film closes the access opening. It is preferred for the film to be adhesively connected and/or welded to the housing, in particular by means of ultrasonic welding. The film is in particular fragile, preferably tearable, so that for example a striking piston of an applicator leads to the film being torn. In such a manner, action of a piston on the punch, preferably indirectly by way of the film, through the access opening is possible. Alternatively to the fragile configuration of the film, it is possible for the film to be configured to be flexible, preferably elastic or resilient. This flexible configuration is in particular resistant to tearing. If in the case of the flexible film action on the punch occurs, the film yields, but still, preferably because of extension, ensures in particular sterile closure of the access opening. Preferably it is also possible for the film to run, or be laid, around the proximal end of the punch and preferably around the side(s) of the punch. If the film runs around the punch in such a manner, it is preferred for the film in the starting position and/or the application position of the support, starting from the distal end of the housing, to arch through the application opening into the interior of the housing and there finally to encompass in particular the punch towards the access opening. Preferably the film comprises an adhesive layer at the distal end, in particular for connecting to an application point.

Alternatively or in addition to the film described above, the sterile barrier comprises in particular a, preferably removable, blister at the application opening and/or a, preferably detachable, film at the application opening. The blister and/or the film closes in particular the application opening. The blister and/or the film is removable, in particular by a user, preferably prior to application, so that application of the microarray can occur. The blister is connected to the housing in particular by adhesion and/or by plugging and/or by means of a fit and/or by means of preferably detachable welding. The film is preferably connected to the housing with a detachable bonded joint and/or by means of, preferably detachable, welding.

The connection on one hand between the film and housing at the access opening and/or on the other hand between the blister and/or film and housing at the application opening is preferably a connection by a material bond and/or an aseptic connection. Thus preferably sterility of the interior of the housing, in particular upon storage, is provided.

It is preferred for the interior of the housing and/or the interior of the blister to have a desiccant and/or an oxygen absorber and/or a protective gas atmosphere. This makes preferably optimum storage of the at least one microarray possible.

It is preferred for the housing and/or the punch to have a desiccant and/or an oxygen absorber. Preferably the material of the housing and/or of the punch is a liquid-absorbing and/or oxygen-absorbing material.

The use of desiccant advantageously makes storage of liquid-sensitive microarrays possible. The use of oxygen absorbers advantageously makes storage of oxygen-sensitive microarrays possible.

It is preferred for the punch to have a punch action surface for, preferably externally, acting on the punch for moving the punch. The punch action surface is preferably arranged on the opposite side of the microarray. In a preferred embodiment, the punch action surface is flat. Instead of the flat embodiment of the punch action surface, it is preferably possible for the punch action surface to have an indentation or a protrusion, in particular a recess facing towards the center of the punch or an elevation facing away from the center of the punch. The indentation or protrusion is preferably embodied as an arch in the one or the other direction. It is particularly preferred for the punch action surface to be made partially spherical or conical or pyramidal towards the inside or outside. A non-flat embodiment of the punch action surface has the advantage that a correspondingly oppositely designed applicator, in particular by means of an applicator punch, acts on the punch action surface for moving the punch and thereby optimum transfer of force and/or directed action is provided.

The method according to the disclosure for producing a support for at least one microarray is in particular a method for producing a support described above. In particular, it is a method for assembling a support for at least one microarray. The method comprises the steps described below, the steps being able to be carried out in any reasonable order. One step of the method consists in manufacturing a housing with an application opening. The manufacturing of the housing preferably takes place by primary forming, such as for example injection-molding, with the application opening being produced in particular directly in the primary forming step, for example by means of the casting mold. A further step of the method for producing a support consists in the manufacturing, in particular the primary forming, of a punch. The steps of manufacturing the housing and of manufacturing the punch can preferably be carried out in any desired order. After the step of manufacturing the punch, the step of connecting a microarray to the punch takes place. Preferably the connection of the microarray and the punch takes place by means of an adhesive join. Further still, the method for producing a support comprises the step of arranging the punch in a movable manner within the housing. The arranging of the punch within the housing takes place in particular in such a manner that the punch in the starting position is fixed relative to the housing, but can be moved due to an action. The step of arranging the punch in a movable manner within the housing takes place after the steps of manufacturing the housing and after the manufacturing of the punch. It is however possible for the step of arranging the punch within the housing to take place before or after the step of connecting the microarray to the punch. In particular, the housing and/or punch can also be produced by means of 3D printing. For production by means of 3D printing it is preferred for direct production of the punch to take place within the housing. In the context of this joint production of punch and housing it is preferred for a supporting structure, in particular a production-related one, to be provided between the punch and housing. Preferably the supporting structure can be removed and/or severed in the method for producing a support. On the other hand, it is however possible for the supporting structure to be retained during production and not to be severed until it is used, in particular applied. In the context of the production by 3D printing, it is thus possible to carry out the arranging of the punch within the housing at the same time as the manufacturing. Furthermore, it is preferably likewise possible to produce a predetermined breaking point, namely the supporting structure, directly in the production by 3D printing.

In a preferred embodiment, the step of connecting the microarray to the punch takes place as a demolding step for the microarray, which is produced in particular by means of a casting process, by means of the punch. Thus preferably the microarray is picked up, in particular from a casting mold, by means of the punch. Preferably the punch and/or the microarray to this end comprises an adhesive layer, so that an adhesive join between the microarray and punch can be produced.

It is preferred for the method to comprise the step of applying a sterile barrier. The sterile barrier preferably comprises at least a film and/or a blister. It is particularly preferred for the sterile barrier to close the application opening and/or an access opening of the housing, preferably in a sterile manner. The sterile barrier is preferably applied by means of gluing and/or welding, in particular ultrasonic welding.

### BRIEF DESCRIPTION OF THE DRAWINGS

The disclosure will be discussed in greater detail below using preferred embodiments with reference to the drawings.

Therein:

FIG. 1 is a schematic perspective view of an embodiment according to the disclosure of a support,

FIG. 2a is a schematic sectional view of the support of FIG. 1 along II. in the starting position,

FIG. 2b shows the support of FIG. 2a in the application position,

FIG. 3 is a schematic sectional view of a further embodiment of a support according to the disclosure,

FIG. 4 is a schematic sectional view of a further embodiment of a support according to the disclosure,

FIG. 5a is a schematic sectional view of a further embodiment of a support according to the disclosure,

FIG. 5b shows the support of FIG. 5b in the application position, and

FIG. 6 is a schematic sectional view of a further embodiment of a support.

### DETAILED DESCRIPTION

Similar or identical components or elements are identified with the same reference numerals in the drawings. In particular for improved clarity, preferably already-identified elements are not provided with reference numerals in all the figures.

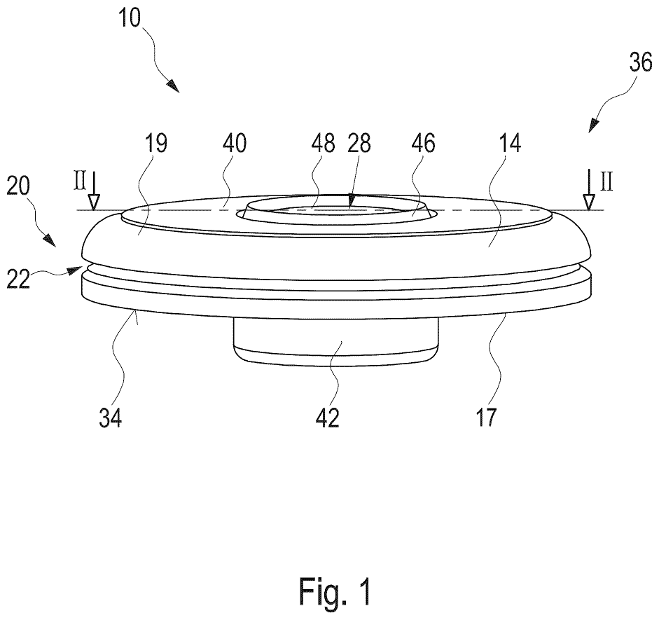

The support 10 of FIG. 1 comprises a housing 14. On the outside, the housing 14 comprises an encircling groove 22 which serves as a connection device for an applicator, not illustrated.

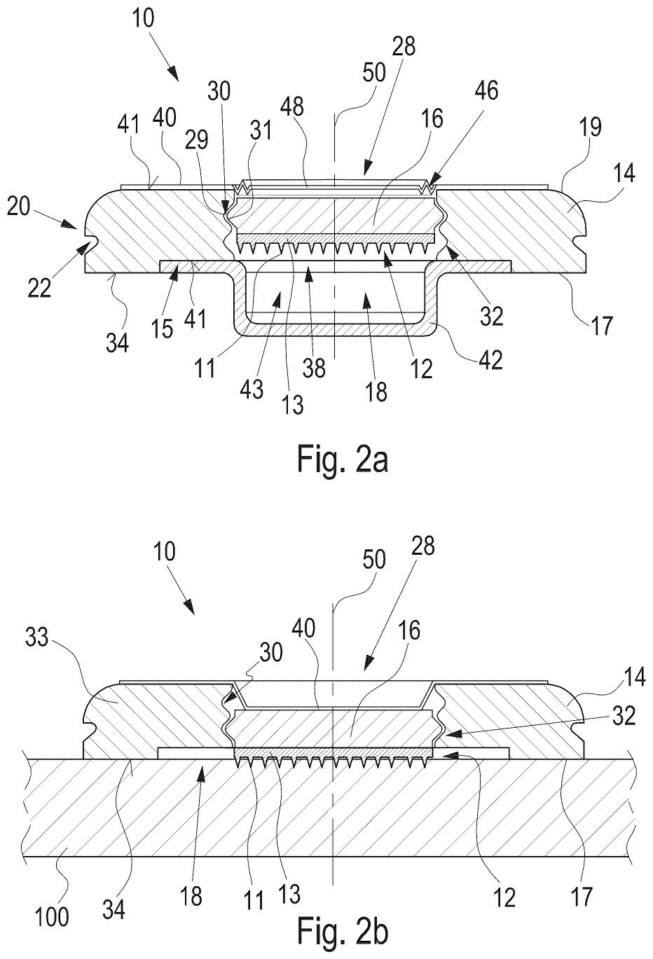

At the proximal end 19, the housing 14 comprises an access opening 28 (see FIG. 2a), through which for example the piston of an applicator, not illustrated, can access, or act on, the interior of the housing 38 (see FIG. 2a).

The access opening 28 is closed from the surroundings by a film 40 which is part of a sterile barrier 36. The film 40 is preferably connected to the housing 14 by means of an adhesive join and/or welded joint 41. The film 40 comprises a flexible region 46, so that for example the piston of an applicator, not illustrated, can distally press in the film interior 48 during access of the piston to the interior of the housing 38. Thus preferably an applicator can move the punch, but sterile isolation from the surroundings is still provided in relation to the illustrated upper side. Instead of the flexible configuration, it is also possible for the film 40, in particular at the region 46, to be configured as fragile, so that for example an applicator can lead to tearing of the film 40 upon movement of the punch 16.

At the distal end 17, a blister 42 is connected to the housing 14. As illustrated in FIG. 2a, for this a cutout 15 which receives the blister 42 can be provided in the housing 14. Additionally or alternatively, the connection between the blister 42 and housing 14 at the connection point 41 can be embodied in particular by means of a detachable adhesive join.

FIG. 2a shows the punch 16 in the starting position. In this case, the punch 16 is fixed in a proximal position relative to the housing by means of release device 30. This fixing takes place by interlocking of the bead 31 of the punch 16 in the groove 29 of the housing 14. The bead 31 and/or groove 29 in this case are preferably embodied to be flexible, with the housing 14 and/or punch 16 being in particular, at least substantially, dimensionally stable.

At the distal end, a microarray 12 is connected to the punch 16. To this end, the patch 13 of the microarray 12 is connected, in particular by adhesion, to the punch. The patch 13 of the microarray 12 comprises distally-extending microneedles 11. In the starting position illustrated in FIG. 2a, the microarray is located within the housing 14. A sterile barrier 36 for isolating the microarray 12 from the surroundings is provided by means of the blister 42 and film 40.

The interior of the housing 38 and/or the interior of the blister 43 may preferably comprise desiccants and/or protective gas and/or oxygen absorbers. This has a positive effect in particular on the sterility and/or shelf life of the microarray.

FIG. 2b shows the support 10 of FIG. 2a in the applied state.

For application, the blister 42 has been taken off or removed, for example by a user. Then the support 10 has been connected by means of an adhesive layer 34 at the proximal end 17 of the housing 14 to an application point 100, which is in particular the skin of a user.

The punch 16 is in the application position. In this case, the punch 16 has moved distally within the housing 14 and in such a manner guided the microarray 12 through the application opening 18 out of the housing 14. The microneedles 11 of the microarray 12 are injected into the application point 100.

The punch 16 is held or fixed in a distal application position by means of a holding device 32. The holding device 32 in the embodiment illustrated has a groove 33 in which the bead 31 of the punch 16 engages. In such a manner, subsequent pressing of the punch 16 and hence longer-lasting application of the microarray 12 is guaranteed.

The movement or deflection of the punch 16 took place by access or action through the access opening 28 on/to the proximal end of the punch 16. This action took place for example by a piston of an applicator, not illustrated, which had been connected to the support 10 in particular by way of the connection device 20. There then took place for example acceleration of a piston of the applicator, which first of all struck the film 40 and distally deflected it owing to its flexible configuration. In particular, the piston indirectly likewise struck the proximal end of the punch 16, as a result of which the release device 30 released the punch 16 for movement. The punch 16 thereupon moved into the distal position illustrated in FIG. 2b and is fixed by the holding device 32.

Instead of the deflection of the punch 16 by means of the applicator, for example deflection or action by way of the finger of a user which presses on the proximal end of the punch 16, preferably indirectly by way of the film 40, is also possible.

The release device 30 is preferably embodied as a desired-force trigger. Thus release of the punch only takes place once at least a desired force is applied, preferably once at least a desired force is applied to the punch. The desired-force trigger may in this case preferably be designed such that the at least necessary desired force corresponds to an in particular optimum application force for puncturing and applying the microarray 12. In the embodiment illustrated, the desired-force trigger is in particular brought about by way of the detachable form-fit between the groove 29 and bead 31.

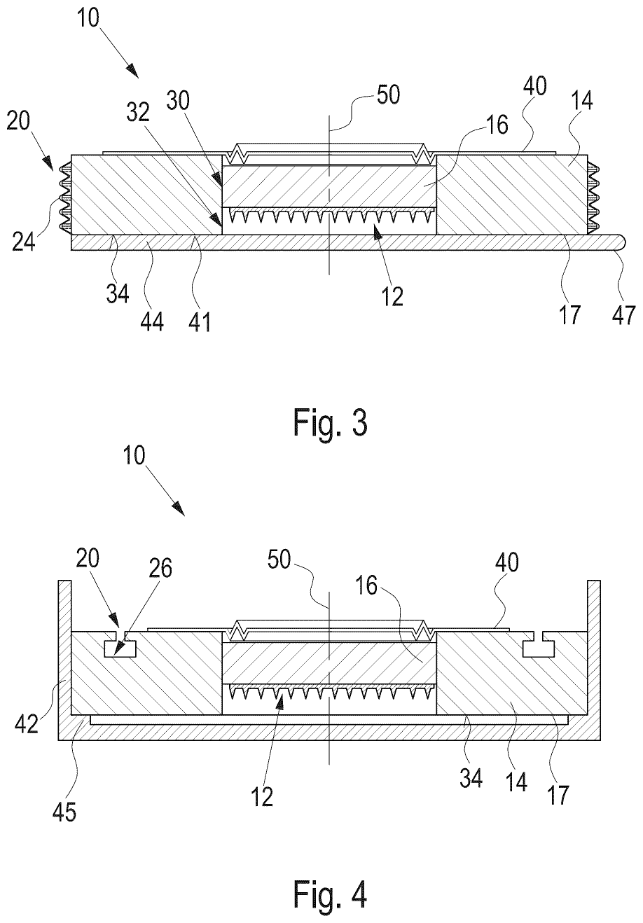

FIG. 3 shows a further embodiment of a support 10 according to the disclosure. The embodiment illustrated is substantially similar to the embodiment of FIG. 2a.

In contrast to the embodiment of FIG. 2a, the release device 30 of the embodiment of FIG. 3 is a fit, in particular interference fit, between the punch 16 and housing 14. Because of the fit, the punch is thus initially held in the starting position. After deflection of the punch 16, the fit likewise brings about the function of the holding device 32 and thus ensures fixing of the punch in the application position.

The connection device 20 comprises a thread 24, to which for example a correspondingly opposite thread of an applicator can be connected.

Likewise, in contrast to the embodiment of FIG. 2a, the embodiment of FIG. 3 does not have a blister, but a film 44. The film 44 is connected at the distal end 17 to the housing 14, in particular by way of an adhesive layer 41. For example, by way of the film tab 47 illustrated the film 44 can be pulled off and be removed in such a manner by a user. The adhesive layer likewise serves as a fixing device 34, so that the support 10 can be connected to an application point therewith.

FIG. 4 shows a further embodiment of a support 10 according to the disclosure. The embodiment is similar to the embodiment of FIG. 3, but has the differences described below.

The embodiment comprises a blister 42 which radially completely encompasses the housing 14. At the proximal end 17, the housing 14 lies against a protrusion 45 extending radially inwards from the blister 42. Viewed from the protrusion 45, the housing 14 has at the distal end 17, radially further to the inside, an adhesive layer 34 which represents a fixing device for fixing the support 10 to an application point. Unlike other embodiments, for example the embodiment of FIG. 3, the adhesive layer 34 does not come into contact with the blister 42, but is spaced apart therefrom.

The blister 42 at the proximal end forms a type of guidance form, for example for an applicator. An applicator can thus for example be pushed into the blister and then be connected to the connection device 20, which as illustrated is embodied as a plug-type connection. Subsequently it is possible for the blister 42, which is preferably merely placed on the housing 14, to be removed. Then an application in the manner of the embodiments described above can take place.

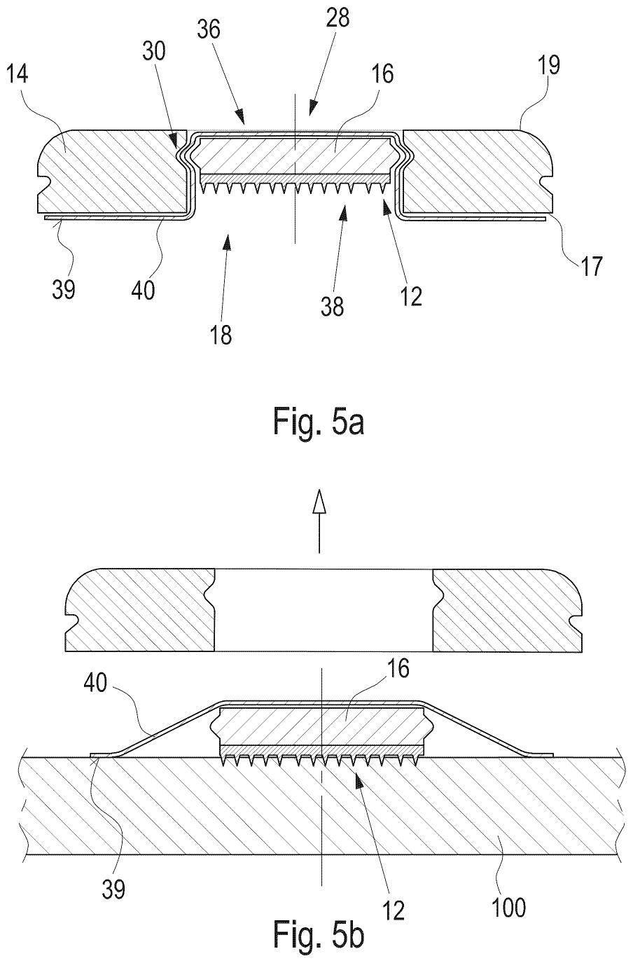

FIGS. 5a and 5b show a further embodiment of a support 10 according to the disclosure. The embodiment does not have a holding device. After movement of the punch 16, preferably with a resultant application of the microarray 12 in the application point 100, the punch 16 and housing 14 are thus no longer connected together or loose from each other. Consequently, the punch 16 and housing 14 can be moved relative to one another.

FIG. 5b accordingly shows removal of the housing 14 from the application site 100, with the punch 16 with the applied microarray remaining on the application point 100.

The sterile barrier 36 illustrated comprises the film 40. In the starting position, as illustrated in FIG. 5a, the film 40 arches from the distal end 17 of the housing 14 through the application opening 18 and encompasses the punch 16 towards the access opening 28. Accordingly, at least proximally, sterility of the interior of the housing 38 or of the microarray 12 is provided. The film 40 comprises preferably distally an adhesive layer 39 so that, as illustrated in FIG. 5b, connection of the film 40 to application point 100 can take place. After or during application (FIG. 5b) the in particular elastic film 40 can on one hand ensure subsequent pressing of the microarray 12 into the application point 100 and/or sterility from the surroundings.

In the embodiment of FIGS. 5a, 5b, it is likewise possible, in particular removably, to provide a blister or a further film for sterility at the distal end, preferably for closing the application opening 18.

The embodiments of the support 10 which are illustrated are formed rotationally symmetrically about axis of rotation 50. However, a different embodiment, for example cuboid, is also possible.

When carrying out the method for producing a support for at least one microarray, it is preferred for the step of connecting the microarray to the punch to take place by means of a punch according to the embodiments illustrated in FIGS. 1 to 4. Preferably, to this end the support 10, prior to the application of a blister 42 or a film 44, with the distal end of the punch 16 is brought into contacting connection with a patch 13 of a microarray 12. The microarray 12 in this case is preferably located in a casting mold used for producing the microarray. The patch 13 of the microarray 12 and/or the distal end of the punch 16 comprises/comprise in particular an adhesive layer, so that once the punch 16 and the microarray 12 have been connected an adhesive connection exists between them. Then the microarray 12 can be taken out of the casting mold by way of the support 10. As a result, preferably the demolding of the microarray 12 takes place by means of support 10.

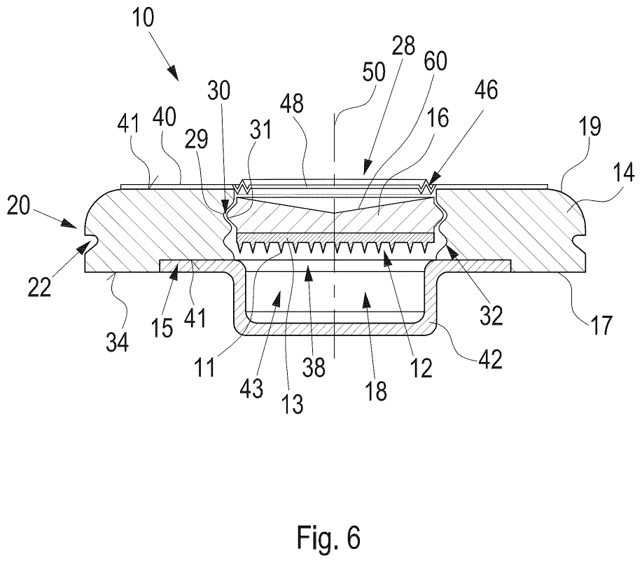

FIG. 6 shows a further embodiment of a support 10. The embodiment illustrated in this case corresponds substantially to the embodiment of FIG. 2a.

Unlike the embodiment of FIG. 2a, the punch action surface 60 of the punch 16 of FIG. 6 is not flat, but embodied as a conical indentation, also referred to as a negative conical shape. For example, a pyramidal or partially spherical indentation is also possible instead of the conical indentation. It is also possible to embody other embodiments of the support 10, for example the embodiments of FIGS. 3 and 4, with a punch action surface 60 defined above.

The conical indentation of the punch action surface 60 of FIG. 6 advantageously causes for example an applicator punch with a correspondingly positive conical shape embodiment to be able to strike the punch action surface 60 and thus cause optimum transfer of force to be provided.

What is claimed is:

1. A support for at least one microarray, with a cylindrical housing, comprising:
a punch arranged in a movable manner within the housing, and
at least one microarray connected to the punch, wherein the housing comprises an application opening for applying the at least one microarray,
the support further comprising a release device between the housing and the punch, wherein the release device fixes the punch in a starting position and releases it for movement upon activation,
wherein the release device comprises a desired-force trigger with at least one of a predetermined breaking point between the housing and the punch, an interference fit between the housing and the punch and/or a detachable form-fit connection between the housing and the punch, wherein the desired-force trigger releases the punch for movement when at least one desired force is applied to the punch.

2. The support according to claim 1, wherein the housing, comprises a detachable or non-detachable connection device for connection to an applicator for moving the punch.

3. The support according to claim 2, wherein the connection device comprises:
a groove, and/or
a bead, and/or
a thread, and/or
a plug-type connection, and/or
a form-fit connecting piece, and/or
an adhesive join, and/or
a magnetic connecting piece.

4. The support according to claim 1, wherein the housing comprises an access opening for an applicator for moving the punch.

5. The support according to claim 1, wherein the housing and/or the punch is at least substantially dimensionally stable.

6. The support according to claim 1, further comprising a holding device, an engagement device and/or snap-in device, for fixing the punch after the release of movement.

7. The support according to claim 1, further comprising a fixing device, an adhesive surface, for fixing the support to an application point.

8. The support according to claim 1, further comprising a sterile barrier for sterile storage of the microarray within the housing, wherein a sterile barrier for closing the application opening and/or the access opening to isolate the interior of the housing from the surroundings.

9. The support according to claim 8, wherein the sterile barrier comprises a fragile or flexible film at the access opening.

10. The support according to claim 8, wherein the sterile barrier comprises a removable blister at the application opening and/or a detachable film at the application opening.

11. The support according to claim 1, wherein the interior of the housing and/or the interior of the blister comprises: a desiccant, and/or an oxygen absorber, and/or a protective gas atmosphere.

12. The support according to claim 1, wherein the punch comprises an inwards or outwards-directed pyramidal or conical or partially spherical punch action surface.

13. The support according to claim 1, wherein the housing and the punch are interlocked with each other to create the detachable form-fit.

14. The support according to claim 1, wherein the detachable form-fit comprises an interlocking bead-and-groove connection.

15. The support according to claim 14, wherein the bead is connected to the housing and the groove is connected to the punch, or wherein the groove is connected to the housing and the bead is connected to the punch.
